(19) United States
(12) Plant Patent Application Publication
(10) Pub. No.: US 2003/0009794 P1
(43) Pub. Date: Jan. 9, 2003

(54) Floribunda rose variety 'POULtry'

(76) Inventors: L. Pernille Olesen, Fredensborg (DK); Mogens N. Olesen, Fredensborg (DK)

Correspondence Address: Poulsen Roser Pacific, Inc., 620 S. Front Street, Central Point, OR, 97502, US

(21) Appl. No.: 10/155,860

(22) Filed: May 24, 2002

Related U.S. Application Data
(63) Continuation of application No. 09/287,294, filed on Mar. 31, 1999.

(51) Int. Cl. A01H 5/00

(52) U.S. Cl. PLT/151

(57) ABSTRACT

A new floribunda rose plant which has abundant, non-fading, red flowers and attractive foliage. The variety successfully propagates from softwood cuttings and is suitable for year round production in commercial glasshouses. This new and distinct variety has shown to be uniform and stable in the resulting generations from asexual propagation.

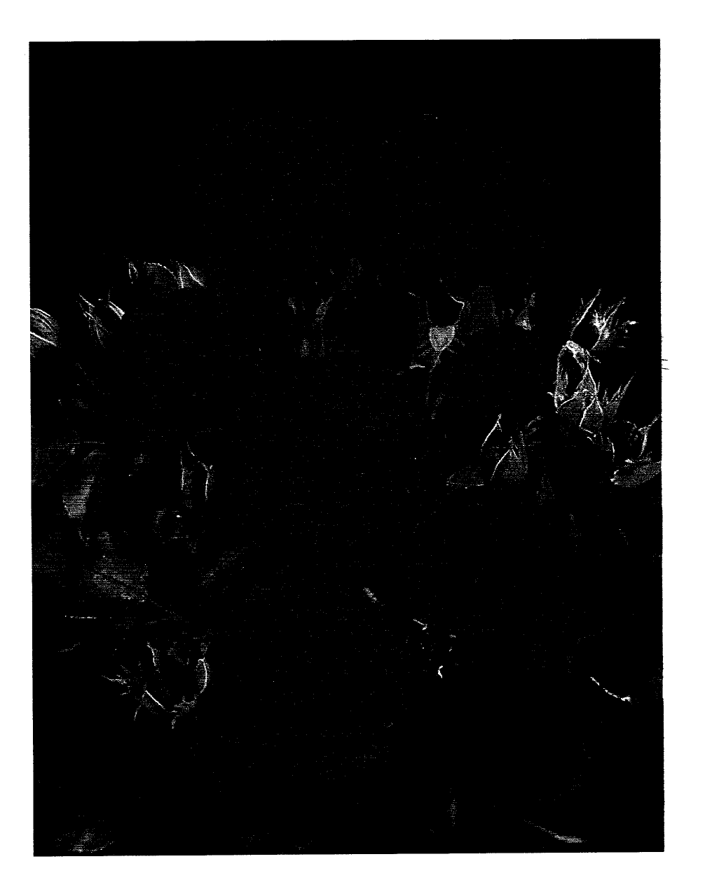

### SUMMARY OF THE INVENTION

The present invention constitutes a new and distinct variety of floribunda rose plant which originated from a controlled crossing between two unnamed, non-patented seedlings. The two parents were crossed and the resulting seeds were planted in a controlled environment. The new variety is named ‘POULtry’.

The new rose may be distinguished from its seed parent, an unnamed seedling created by the same inventors, by the following:

The unnamed seed parent has semi-double dark red flowers, while ‘POULtry’ has very double, dark red flowers.

The new variety may be distinguished from its pollen parent, an unnamed seedling created by the same inventors, by the following combination of characteristics:

1. The pollen parent is a low growing, compact floribunda, while ‘POULtry’ is an upright, taller and more vigorous growing floribunda.

2. The pollen parent has pink flowers, while ‘POULtry’ has dark red flowers.

The objective of the hybridization of this rose variety for commercial greenhouse culture was to create a new and distinct variety with unique qualities, such as:

1. Uniform and abundant flowers;

2. Vigorous and compact growth;

3. Year-round flowering under glasshouse conditions;

4. Suitability for production from softwood cuttings in pots;

5. Durable flowers and foliage which make a variety suitable for distribution in the floral industry.

This combination of qualities is not present in previously available commercial cultivars of this type and distinguish ‘POULtry’ from all other varieties of which we are aware.

As part of their rose development program, L. Pernille Olesen and Mogens N. Olesen germinated the seeds from the aforementioned hybridization and conducted evaluations on the resulting seedlings in a controlled environment in Fredensborg, Denmark.

‘POULtry’ was selected by the inventors as a single plant from the progeny of the hybridization in spring of 1987.

Asexual reproduction of ‘POULtry’ by cuttings and traditional budding was first done by L. Pernille and Mogens N. Olesen in August, 1987, at their nursery in Fredensborg, Denmark. This initial and other subsequent propagations conducted in controlled environments have demonstrated that the characteristics of ‘POULtry’ are true to type and are transmitted from one generation to the next.

### BRIEF DESCRIPTION OF THE DRAWING

The accompanying color illustration shows as true as is reasonably possible to obtain in color photographs of this type, the typical characteristics of the buds, flowers, leaves, stems, and a plant of ‘POULtry’. Specifically illustrated in SHEET 1:

1. Stem or entire plant showing branching and the attachment of leaves, buds, and peduncles;

2. Flower bud, partially opened bud, and open bloom;

3. Flower petals, detached;

4. Sepals, receptacle, and pedicel;

5. Bare stem exhibiting thorns;

6. Leaves.

### DETAILED DESCRIPTION OF THE VARIETY

The following is a description of ‘POULtry’, as observed in its growth in a field nursery in Jackson County, Oreg., on plants aged eighteen months. Color references are made using The Royal Horticultural Society (London, England) Colour Chart, 1995, except where common terms of color are used.

For a comparison, the nearest existing rose variety is ‘POULander’, a patented variety by the same inventors described and illustrated in U.S. Plant Pat. No. 6,265 and issued on August 30, 1988. Chart 1 details several physical characteristics of the instant variety and the comparison variety.

CHART 1

| | ‘POULtry’ | ‘POULander’ |
|---|---|---|
| Bud color at ¼ | Red-Purple Group 60B | Red Group 46B-46C |
| open | | |
| Upper surface of | Red Group 53A | Red Group 46B |
| petal, open bloom | | |
| Reverse surface of | Red Group 53B | Red Group 53C |
| petal, open bloom | | |
| Petalage | 70-80 petals | 20 petals |

Parents: Unnamed seedling x Unnamed seedling.

Classification:

Botanical.—Rosa hybrida.

Commercial.—Floribunda.

### FLOWER AND FLOWER BUD

Blooming habit: Recurrent.

Flower bud:

Size.—Upon opening, 20 mm-22 mm in length from base of receptacle to end of bud.

Bud form.—Short and round.

Bud color.—As sepals unfold, Red-Purple Group 59A.

Sepals.—Green Group 143B. Moderate foliaceous appendages on three of the five sepals. Surfaces of other sepals slightly pubescent. A limited number of stipitate glands on all of the sepals. Sepals are 20 to 25 mm long and 10 to 13 mm wide. The sepal's apex is cirrose in shape with a flat base.

Receptacle.—Surface: Smooth, glabrous. Shape: Funnel. Size: Small. 10 mm (h)×8-10 mm (w). Color: Yellow-Green Group 144B.

Peduncle.—Surface: Scant stipitate glands. Length: 50-70 mm average length. Color: Yellow-Green Group 144A. Strength: Strong.

Borne.—Multiple buds per stem. Generally with 4-8 buds per flowering stem.

Flower bloom:

Size.—Medium for a floribunda rose. Average flower diameter is 50-80 mm when open.

Form.—Shape of flower when viewed from the side: Upon opening, upper part: Flattened convex. Upon opening, lower part: Convex. Open flower, upper part: Convex. Open flower, lower part: Concave. Petals: Slightly reflexing. Petalage: Very double. Average range: 70-80.

Color:

Upon opening.—Outermost petals: Upper Surface: Red-Purple Group 60B. Reverse Side: Red-Purple Group 60B. Innermost petals: Upper Surface: Red-Purple Group 60A. Reverse Side: Red-Purple Group 60B. Basal petal spots: Outermost petals: Outer Side: Green-White Group 157C. Inner Side: Green-White Group 157B. Innermost petals: Outer Side: Green-White Group 157B. Inner Side: Green-White Group 157B.

After opening.—Outermost petals: Upper Surface: Red Group 53A. Reverse Side: Red Group 53B. Innermost petals: Upper Surface: Red Group 53B. Reverse Side: Red Group 53B. Basal petal spots: Outermost petals: Outer Side: Green-White Group 157D. Inner Side: Green-White Group 157A. Innermost petals: Outer Side: Green-White Group 157D. Inner Side: Green-White Group 157C. Texture: Petal surface has velvety silver sheen which tends to give a pink tone to the individual petals.

General Tonality: On open flower Red Group 53A. No change in the general tonality at the end of the third day. Petals:

Petal reflex.—Petal reflex is weak.

Petal edge.—Entire with some undulation.

Petaloids.—3 to 4. Petaloids are 5 to 7 mm long and 3 to 5 mm wide. Petaloid coloration is Red Group 53B on both outer and inner sides. Texture is smooth.

Fragrance.—Light fruit scent.

Duration: Flowers last on the bush from 3-7 days. Vase life of the cut flowers is 2-4 days.

Thickness.—Thick.

Shape.—Rounded obovate.

Form.—Reflexed slightly.

Arrangement.—Not imbricated.

Reproductive Organs:

Pistils.—Quantity: 25 to 30. Length: 7 to 10 mm.

Pollen.—Color: Greyed-Orange Group 167D. Quantity: Average.

Filament.—Size: 8 to 10 mm. Color: Yellow-Green Group 157B with some intonation of Yellow Group 13C. Quantity: 25 to 30 Length: 5 to 7 mm.

Anther.—Color: Yellow Group 13. Length: 2 mm

Stigmas.—Superior in location to anthers. Color: Yellow Group 162C.

Styles.—Color: Yellow-Green Group 157B.

Hips.—None observed.

### PLANT

Plant growth: Upright and bushy plant. Typically, with foliage from top to bottom of the plant After two growing seasons, when grown as a budded field grown plant on Rosa multiflora understock, the average height of the plant is 90 to 120 cm with an average width of 80 to 100 cm. The typical height of the variety is 90-120 cm with an average width of 80-100 cm.

Stems:

Color.—young wood: Green Group 143B-143C. Older wood: Green Group 139C.

Thorns.—Incidence: Numerous small thorns on the flowering stems. Size: Average length: 4 mm -10 mm. Color: Mature thorns Yellow-Green Group 152A. Immature thorns Yellow-Green Group 153B. Shape: Concave.

Surface.—Young wood: Smooth. Older wood: Smooth.

Plant foliage: Normal number of leaflets on normal leaves in middle of the stem: 5 leaflets.

Leaf size.—Medium. 10-11 mm (1) ×8 mm (w).

Abundance.—Very abundant.

Color.—Upper Leaf Surface: Green Group 137A. Lower Leaf Surface: Green Group 147B. Juvenile foliage: Greyed-Purple Group 183C. Anthocyanin intonation: Location: New growth and peduncles. Color: Greyed-Purple Group 183C.

Plant leaves and leaflets:

Stipules.—Present. Size: Large. 20 mm (1) ×6 mm (w). Color: Yellow-Green Group 144C. Stipitate glands: At the margin of the stipule.

Petiole.—Length: 25 mm. Color: Yellow-Green Group 144C. Underneath: Margins: Scant stipitate glands. Prickles: Few or none.

Rachis.—Color: Yellow-Green Group 144A. Underneath: Margins: Have scant stipitate glands which are dark rose in color. Rachis has red coloration where leaves are attached.

Leaflet.—Edge: Serrated. Shape: Orbicular. The leaflet's apex is acuminate. The leaflet's base is rounded. Arrangement: The leaflets are arranged in an odd-pinnate formation. Venation: The leaflets are veined in a reticulate pattern. Texture: Upper side of leaflet is glossy. Lower side of leaflet is matte. Leaflet is thick.

Disease resistance:

Excellent resistance to mildew, rust, and Botrytis under normal growing conditions in Jackson County, Oreg. The resistance to black spot is good, with the plant showing some symptoms when the disease is present in the field, but fewer symptoms than other varieties in the same field.

Winter hardiness:

‘POULtry’ has been found to be resistant to damage from cold in USDA Zone 8 and USDA Zone 7.

1. A new and distinct variety of rose plant of the floribunda class, substantially as herein illustrated and described as a distinct and novel rose variety due to its abundant, red flowers, vigorous and compact growth, year round flowering under glasshouse conditions, suitability for production from softwood cuttings in pots, and durable flowers and foliage which make the variety suitable for distribution in the floral industry.
